(19) United States
(12) Patent Application Publication
(10) Pub. No.: US 2015/0240294 A1
(43) Pub. Date: Aug. 27, 2015

(54) METHOD FOR DETECTING METHICILLIN-RESISTANT STAPHYLOCOCCUS AUREUS (MRSA) STRAINS

(71) Applicant: QIAGEN Hamburg GmbH, Hilden (DE)

(72) Inventor: Arnim Wiezer, Hamburg (DE)

(21) Appl. No.: 14/657,148

(22) Filed: Mar. 13, 2015

Related U.S. Application Data
(63) Continuation of application No. 13/143,050, filed on Jun. 30, 2011, now abandoned.

(30) Foreign Application Priority Data
Dec. 30, 2008 (EP) 10 2008 063 360.7

(51) Int. Cl. C12Q 1/68

(52) U.S. Cl. CPC C12Q 1/689

(57) ABSTRACT

The present invention relates, inter alia, to a method for detecting methicillin-resistant Staphylococcus aureus (MRSA) strains, wherein one of the following method variants is carried out:
variant A
a) chromosomal DNA of S. aureus is isolated from the sample by means of a genome probe, and
b) a nucleotide sequence which is specific for MRSA is detected in the isolated DNA;
or
variant B
a) DNA is isolated from the sample, wherein the isolation makes use of a genome probe which is specific for an MRSA nucleotide sequence, preferably an MRSA resistance gene, and
b) the DNA isolated in step a) is tested for specific sequences of S. aureus.
   In addition, suitable kits for carrying out the corresponding methods are provided.

The present invention relates to a method for detecting MRSA in a sample, and to suitable kits and means for carrying out corresponding methods.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 771025_{—}402C1_{—SEQUENCE}_LISTING.txt. The text file is 3 KB, was created on Mar. 13, 2015, and was submitted electronically via EFS-Web.

### BACKGROUND OF THE INVENTION

Staphylococcus aureus (S. aureus ) is a Gram-positive bacterium which colonizes the skin, and can be found in the frontal part of the nasal cavity in about 25-30% of healthy people. It can cause a range of diseases in humans, for example wound infections, pneumonia, sepsis, and endocarditis. To treat S. aureus infections, use is preferably made of beta-lactam antibiotics, examples of such beta-lactam antibiotics being methicillin, oxacillin, dicloxacillin, and flucloxacillin.

After the introduction of methicillin in the sixties, the emergence of S. aureus strains which were resistant to methicillin, known as “methicillin-resistant Staphylococcus aureus strains” or, for short, “MRSA” strains, was observed. Since the eighties, MRSA has been a considerable clinical and epidemiological problem in hospitals, since MRSA is resistant to all beta-lactam antibiotics, including penicillin, cephalosporin, carbapenem, and monobactam, which are principally used to treat S. aureus infections.

MRSA infections can be treated only with relatively expensive antibiotics having higher toxicity, but in many cases the end result is still death of the afflicted persons.

Methicillin resistance is caused by the uptake and the incorporation of an exogenous gene, the mecA gene. The mecA gene encodes an additional beta-lactam—resistant penicillin binding protein (PBP) which is referred to as PBP 2a or PBP2′. This takes over the biosynthetic functions of the normal PBPs when the cell is exposed to beta-lactam antibiotics. The mecA gene is not present in MSSA strains (“methicillin-susceptible S. aureus”), but occurs as a highly conserved gene in many other staphylococci species, for example Staphylococcus epidermidis, S. haemolyticus, S. saprophyticus, S. capitis, S. warneri, S. sciuri, and S. caprae.

The mecA gene is part of a large mobile genetic element which is referred to as “Staphylococcus cassette chromosome mec” (SCCmec) and is taken up by a methicillin-susceptible S. aureus strain (MSSA) which is thereby converted into an MRSA strain. This cassette is integrated in the immediate vicinity of the bacterial origin of replication.

SCCmec is characterized by the presence of terminal inverted and direct repeats, a set of site-specific recombinase genes, and the mec gene complex (Hiramatsu et al., 2002, Int. J. Med. Microbiol. 292: 67-74). The SCCmec DNA is integrated into the MSSA chromosome at a specific site, and this site is located at the 3′ end of an open reading frame (ORF), the function of which is not known and which is referred to as orfX.

To date, up to seven different SCCmec types and multiple variants thereof have been described. The various SCCmec elements differ in, for example, the genes providing antibiotic resistance to non-beta-lactam antibiotics.

Since MRSA can be transmitted very easily from hospital personnel to patients, the monitoring of MRSA in hospitals constitutes a considerable problem worldwide. Therefore, there is a great need for fast and simple screening methods with which MRSA can be detected or identified in order to minimize its spread and to improve the diagnosis or treatment of the afflicted patients, respectively.

Various methods for detecting MRSA have been proposed and have also been used. The early molecular tests were based on the detection of an S. aureus-specific gene and/or mecA. The disadvantage of these methods is that they are not suitable for the direct detection of MRSA from samples such as, for example, a nasal swab, since S. aureus bacteria first have to be specifically enriched, since the samples may contain further staphylococci which likewise contain mecA.

A test method based on separate PCRs detects mecA and a nucleotide sequence specific for S. aureus . But also here the problem of mecA being present in different staphylococci arises, such that for example, samples which contain MSSA and S. epidermidis (mecA+) are also identified as false positives. Therefore, the direct detection of the mecA gene in a sample cannot be used as proof of the presence of MRSA. This method is therefore only meaningful when a bacterial strain from the sample of the patient has first been cultured and has then been identified as S. aureus.

Patent EP 0 887 424 discloses a method for detecting the presence of MRSA, in which a reaction with a sample is carried out, by combined use, as primer and/or as probe, of: (1) part of a mecA DNA, which is an integrated non-inherited DNA which is present on a chromosome of the MRSA and carries a mecA gene, and (2) part of a nucleotide sequence of chromosomal DNA surrounding the integrated DNA.

Real-time PCR approaches use the SCCmec insertion site (orfX) and chromosomal DNA surrounding it to detect MRSA. The SCCmec right extremity sequences (SRE) vary in the various SCCmec types and are adjacent to chromosomal DNA of S. aureus , after the cassette has been integrated into the S. aureus genome. In the method which is disclosed in the international patent application WO 02/099034, use is made of specific mec-side primers and MSSA-side primers in a PCR reaction in order to detect the integration of the SCCmec cassette into the MSSA genome. PCR products are only obtained when the cassette has actually integrated. Most notably, PCR products are not obtained when MSSA without SCCmec is present or when another staphylococcal strain is present which contains mecA but is not MSSA. A disadvantage of this method is that new types of SCCmec may not be detected, viz. if the SRE sequences deviate from the known sequences, so that the mec-side primers can no longer bind and therefore no PCR product is formed, although MRSA is present. A further disadvantage is that, in a few cases, the SCC cassette does not contain mecA, and thus false-positive signals are produced in this case, since S. aureus is detected which, although containing an SCC cassette, is nevertheless not methicillin-resistant.

In prior art, there is therefore a need for further methods for detecting MRSA. It is therefore an object of the invention to provide such methods.

### SUMMARY OF THE INVENTION

The present invention is based on the finding that, for the detection of MRSA in a sample, it is advantageous to isolate target DNA from the rest of the sample in a first step and then to carry out in a second step a detection which, in combination with the first step, is MRSA-specific. The combination of these two steps permits efficient detection of MRSA even in mixed samples, i.e., in samples which contain not only the target DNA but also non-target DNA. Examples of non-target DNA are human DNA or DNA from other organisms which, for example, are not MRSA.

According to a first aspect of the present invention, there is provided a method for detecting MRSA in a sample. The method according to the invention can be carried out as per two variants which are both based on the above mentioned principle.

According to a first embodiment of the method according to the invention (variant A), there is provided a method for detecting MRSA in a sample, wherein
a) chromosomal DNA of S. aureus is isolated from the sample by means of a genome probe, and
b) a nucleotide sequence which is specific for MRSA is detected in the isolated DNA.

In step a), a genome probe is used to isolate chromosomal DNA of S. aureus from the sample. In this variant, the S. aureus DNA is the target DNA. By means of the genome probe, it is possible to remove the S. aureus DNA from the non-target DNA in the sample. What is achieved as a result is that chromosomal S. aureus DNA is specifically isolated and enriched from the sample and is separated from the DNA of other organisms which might distort the test result (for example, mecA-carrying staphylococci, such as, for example, Staphylococcus epidermidis, S. haemolyticus, S. saprophyticus, and S. capitis; see above). In step b), a nucleotide sequence which is specific for MRSA (for example the mecA gene) is then detected in the target DNA isolated in step a). This specific detection step makes it possible to test whether MRSA DNA is actually detectable in the isolated S. aureus DNA and whether the analyzed sample is positive with regard to MRSA. The combination of steps a) and b) therefore allows the efficient detection of MRSA in a sample.

According to a second embodiment of the method according to the invention (variant B), there is provided a method for detecting MRSA in a sample, wherein
a) DNA is isolated from the sample, wherein the isolation makes use of a genome probe which is specific for an MRSA nucleotide sequence, preferably an MRSA resistance gene, and
b) the DNA isolated in step a) is tested for specific sequences of S. aureus.

In step a), a genome probe is used to isolate, from the sample, DNA which contains an MRSA resistance gene. In this variant, the DNA which has an MRSA nucleotide sequence (preferably a resistance gene, such as mecA) is the target DNA. By means of the genome probe, this target DNA is again separated from the non-target DNA in the sample. A non-target DNA in this variant of the method according to the invention would be, for example, DNA which has no MRSA resistance gene (for example, human DNA, or DNA of MSSA strains). As a result of the isolation by means of the genome probe specific for an MRSA nucleotide sequence, what is likewise achieved is that the target DNA is specifically isolated and concentrated from the sample and can be separated from the non-target DNA. As explained above, there are, however, also strains which carry MRSA sequences, such as the mecA gene for example, but which are not S. aureus bacteria and accordingly not MRSA. Therefore, according to variant B, in step b), it is tested whether the DNA isolated in step a) has S. aureus sequences and whether the analyzed sample is positive with regard to MRSA. For this purpose, the DNA isolated in step a) is tested for the presence of sequences which are specific for S. aureus. The combination of steps a) and b) therefore enables MRSA to be detected in a sample in an efficient manner.

According to a further aspect of the present invention, there is provided a kit for detecting MRSA in a sample. This kit is suitable for carrying out the method according to the invention.

According to a first embodiment of the kit (variant A), there is provided a kit for detecting MRSA in a sample, comprising at least the following components:
a) at least one genome probe which is suitable for isolating DNA of S. aureus , and
b) means for detecting a nucleotide sequence which is specific for MRSA.

This kit is suitable especially for carrying out the method according to the invention as per variant A. The genome probe present in the kit according to the invention enables the isolation of the S. aureus target DNA, which is especially advantageous for mixed samples. In addition, the kit comprises means for detecting a nucleotide sequence which is specific for MRSA. Details concerning the interaction of the respective elements have already been described above in conjunction with the corresponding method (variant A); reference is made to the corresponding embodiments. Suitable means are well known to a person skilled in the art and are not only probes but also, for example, oligonucleotides or oligonucleotide mimetics which enable appropriate detection by means of PCR.

According to a further embodiment of the kit (variant B), there is provided a kit for detecting MRSA in a sample, comprising at least the following components:
a) at least one genome probe which is suitable for isolating, from a sample, DNA having an MRSA nucleotide sequence, preferably an MRSA resistance gene, and
b) means for detecting S. aureus-specific DNA sequences.

This kit is suitable especially for carrying out the method according to the invention as per variant B. The genome probe present in the kit according to the invention enables the isolation of the target DNA which has an MRSA nucleotide sequence, preferably an MRSA resistance gene, and this is advantageous especially for mixed samples. In addition, the kit comprises means for detecting S. aureus-specific DNA sequences. Details concerning the interaction of the respective elements have already been described above in conjunction with the method (variant B); reference is made to the corresponding statements. Suitable means for the detection of DNA sequences are well known to a person skilled in the art and are not only probes but also, for example, oligonucleotides or oligonucleotide mimetics which enable appropriate detection by means of PCR.

Further objects, features, details, and embodiments of the present invention can be found in the following description and in the accompanying claims. However, the following description serves only to illustrate the present invention.

### BRIEF DESCRIPTION OF THE DRAWING

FIG. 1 shows the results of PCR reactions according to the Example. The curve on the left is from a reaction using the S. aureus DNA purified by means of a conventional method as template. The curve on the right is from a reaction using the S. aureus DNA purified according to the method provided herein as template.

### DETAILED DESCRIPTION OF THE INVENTION

The object of the invention is achieved, inter alia, by the method according to the invention as claimed in claim 1 as per variant A.

The invention therefore provides, according to one aspect, a method for detecting MRSA in a sample, wherein:
a) chromosomal DNA of S. aureus is isolated from the sample, and
b) a nucleotide sequence which is specific for MRSA is detected in the isolated DNA.

In a preferred embodiment, the nucleotide sequence is a gene. In a further preferred embodiment, the gene is a resistance gene, and in a particularly preferred embodiment, the resistance gene is mecA.

“Specific for MRSA” in the present case means that the nucleotide sequence is present in MRSA but not in MSSA strains.

The method according to the invention has the advantage that the isolation of the S. aureus DNA carried out in the first step ensures that, in the case of an MRSA-specific nucleotide sequence, more particularly the resistance gene mecA, being detected in the second step, it is actually MRSA. By means of the method according to the invention, it is therefore possible to specifically detect MRSA even in mixed samples without the need to culture the bacteria. The risk of detecting false positives which are caused by the detection of MSSA and the mecA gene from another staphylococcal strain is therefore reduced in the method according to the invention by the combination of the target DNA-specific isolation in step a) and the MRSA-specific detection in step b). Also, the method according to the invention is not reliant on the use of the SRE sequences, and so new SCCmec sequences having unknown SRE sequences cannot be missed.

The chromosomal DNA can be released before or at the same time as isolation from the bacterial cells. A complete disruption of the bacterial cells is advantageous for isolation of the chromosomal DNA and therefore preferred.

The chromosomal S. aureus DNA can be isolated by any method known in the prior art. In a preferred embodiment, the chromosomal S. aureus DNA is isolated by means of a specific genome probe. The genome probe is a nucleotide sequence which is complementary to a particular part of the chromosomal S. aureus DNA, this part being specific to S. aureus. Such DNA sequences specific to S. aureus can be determined by a person skilled in the art, for example by genome comparisons using bioinformatics programs.

In a preferred embodiment, the genome probe is complementary to a segment of the chromosomal S. aureus DNA which is in the vicinity of the SCCmec insertion site. This ensures that the SCCmec cassette, in the case of it being integrated into the genome, this always being the case for MRSA, is likewise isolated, even when fragments are formed during the isolation of the chromosomal S. aureus DNA.

According to one embodiment, the S. aureus-specific genome probe is selected such that it is complementary to a segment of the chromosomal S. aureus DNA which is within a range of 20 kb, preferably 10 kb, particularly preferably 5 kb, of the SCCmec insertion site. The closer the genome probe-binding segment to the SCCmec insertion region, the lower the risk, in the case of DNA fragmentation during DNA isolation, of the SCCmec cassette and thus the MRSA-specific nucleotide sequence to be detected not (at least in part) being located on the fragment which is bound to the genome probe and can be detected accordingly in step b). The selection of a segment in the vicinity of the SCCmec insertion site therefore increases the probability, even in the case of DNA fragmentation, of the MRSA-specific nucleotide sequence to be detected (if present in the sample) being, at least in part, located on an S. aureus DNA fragment which has bound to the genome probe and has thus been isolated.

According to one embodiment, use is made of a genome probe which is complementary to a segment of the chromosomal S. aureus DNA which is in the vicinity of the mecA gene and is S. aureus-specific. Preferably, the genome probe is complementary to a segment which is within a range of 25 kb, preferably 10 kb, particularly preferably 5 kb, of the mecA gene and is S. aureus-specific. This variant is advantageous if the mecA gene is detected as an MRSA-specific nucleotide sequence. In the case of DNA fragmentation during DNA isolation, this design increases the probability that a DNA fragment carrying (at least in part) the mecA gene is bound to the genome probe.

According to one embodiment, the genome probe used in the method according to the invention as per variant A has at least the following sequence: ATGAAAGCTTTATTACTTAAAACAAGTGTATGGCTCGTTTTGCTTTTTAGTGTAATGG GATTATGGCAAG (SEQ ID NO 7). As shown by the examples, this genome probe is suitable for isolating chromosomal S. aureus DNA from a sample. Thus, the S. aureus genome segment selected for this genome probe is a suitable target for isolating S. aureus-specific DNA in which nucleotide sequences which are specific for MRSA can be subsequently detected as well. Accordingly, use can also be made of an S. aureus-specific genome probe which is complementary to a segment of the chromosomal S. aureus DNA which is within 1 kb, preferably within 500 bp, particularly preferably within 250 bp, of the region of the S. aureus chromosome to which the sequence having SEQ ID NO 7 is complementary. However, it is also possible to design other suitable probes on the basis of available database entries for S. aureus and to check them for their efficiency.

In a preferred embodiment, a mixture of different genome probes is used for isolating the chromosomal S. aureus DNA. This ensures that, despite possible fragmentation by the chromosomal S. aureus DNA, the corresponding fragments which contain the MRSA-specific nucleotide sequence, more particularly the MRSA-specific resistance gene, can be isolated and can thus be detected by the method according to the invention.

According to one embodiment, the genome probe is ≧20 nucleotides and preferably ≦100 nucleotides in length. This range of lengths has been found to be particularly suitable not only for specifically binding S. aureus DNA, but also for avoiding undesired folding and hybridizations of the genome probe.

According to one embodiment, the genome probe is bound to a support. The genome probes used for the isolation can be bound to any surfaces of suitable supports, such as, for example, magnetic beads, spin column filters, etc. The isolation of the DNA and/or the binding of the target DNA to the genome probe are then carried out using methods which are well known in the prior art. Various possibilities are known in the prior art for binding genome probes to the surfaces of support materials. Accordingly, a detailed description is not needed in this regard; nevertheless, some variants ought to be mentioned. For instance, the genome probe can be bound to the support via, for example, a spacer or linker, for example a nucleotide spacer. This has steric advantages, in particular when long DNA fragments are to be isolated.

According to one embodiment, the genome probe is covalently bonded to a support. This embodiment is advantageous when the DNA is purified directly from the biological sample using the genome probe and, accordingly, no general DNA purification step precedes the isolation of the target DNA in step a).

In addition, noncovalent coupling systems can be used for binding the genome probe to the support (for example, binding via streptavidin/biotin). Appropriate systems are well known in the prior art and also commercially available.

According to one embodiment, the genome probe is contacted with the DNA in a first step and the support is added in a second step, so that the genome probe can be bound to the support. A corresponding system can be used when, for example, the genome probe is non-covalently bound to the support, and has also been used in the example. However, the genome probe can also be present bound to the support before it is contacted with the DNA.

For the isolation of the nucleic acid from the sample, methods known in the prior art can be used. In the prior art, there are, for example, many known methods for nucleic acid purification which consist of the combination of a solid phase with a chaotropic buffer. A nucleic acid isolation method suitable for a multiplicity of different applications is disclosed in, for example, U.S. Pat. No.5,234,809. It describes a method for isolating nucleic acids from nucleic acid-containing starting materials by the incubation of the starting material with a chaotropic buffer and a DNA-binding solid phase. The chaotropic buffers achieve, if necessary, both the lysis of the starting material and also the binding of the nucleic acids to the solid phase. The method is highly suitable for isolating nucleic acids from smaller sample amounts. A method based on a similar principle is also described in WO93/11221. Such methods for unspecific DNA purification can precede the method according to the invention.

According to one embodiment of the method according to the invention, total DNA is isolated from the sample in an upstream purification step before, as per variant A, the chromosomal S. aureus DNA is isolated from the prepurified total DNA by means of the genome probe in step a). According to another embodiment of the method according to the invention, the chromosomal S. aureus DNA is directly isolated from the biological sample by means of the genome probe. According to this embodiment, no general DNA purification step precedes the isolation of the target DNA in step a). The same applies to the method according to the invention as per variant B, which will be described in detail below.

The isolation of the chromosomal S. aureus DNA in step a) of the method according to the invention as per variant A is carried out by, for example, contacting the biological sample with the genome probes, resulting in the chromosomal S. aureus DNA binding thereto. Suitable conditions for binding are known in general to a person skilled in the art. When, for example, magnetic beads are used, they are contacted with the biological sample, which, if necessary, is treated beforehand or at the same time such that the DNA present in the bacterial cells is released therefrom, resulting in the chromosomal S. aureus DNA binding to the genome probes on the beads. These can then be separated from the rest of the sample and the, if applicable, likewise released non-target DNA using a magnet, washed, and the bound chromosomal S. aureus DNA can be subsequently eluted or released from the genome probe. Afterwards, the MRSA-specific nucleotide sequence is detected in step b). The same applies to the method according to the invention as per variant B, which will be described in detail below.

In a preferred embodiment, a resistance gene is detected as an MRSA-specific nucleotide sequence, and in a particularly preferred embodiment, more than one MRSA-specific resistance gene can be detected. In a particularly preferred embodiment, the resistance gene is mecA.

The MRSA-specific nucleotide sequence can be detected using any nucleic acid detection method known in the prior art.

In a preferred embodiment, the MRSA-specific nucleotide sequence, more particularly the resistance gene, such as mecA for example, is detected by means of PCR. In a particularly preferred embodiment, the detection is carried out by means of real-time PCR. Primers and detection probes suitable for PCR and real-time PCR can be easily prepared by a person skilled in the art. For this purpose, use can be made of not only oligonucleotides but also oligonucleotide mimetics, such as PNAs or LNAs for example. However, other detection methods are also conceivable, such as, for example, the use of labeled probes which can detect the MRSA-specific nucleotide sequence. These probes may likewise be oligonucleotides or oligonucleotide mimetics. Appropriate detection methods are well known in the prior art and therefore do not require a detailed description. Suitable primers are shown in the example (SEQ ID NO 9 to 11). As shown in the example, these enable the PCR detection of mecA. The primers as per SEQ ID NO 9 and 10 enable regular PCR detection; the probe as per SEQ ID NO 11 enables, in combination with the other two primers, detection by means of real-time PCR.

The object of the invention is further achieved by the method according to the invention as claimed in claim 1 as per variant B.

According to this further embodiment of the invention, there is provided a method for detecting MRSA in a sample, wherein
a) DNA is isolated from a sample, wherein the isolation makes use of a genome probe which is specific for an MRSA nucleotide sequence, preferably an MRSA resistance gene, and
b) the DNA isolated in step a) is tested for specific sequences of S. aureus.

This method likewise enables fast and specific detection of MRSA in a mixed sample. By means of step a), the DNA which has nucleotide sequences, more particularly resistance genes, that are also found in MRSA is first isolated. Preference is given to this being mecA. Since not only S. aureus but also other staphylococci contain MRSA-specific nucleotide sequences, such as mecA for example, the DNA of those bacteria having the corresponding MRSA-specific nucleotide sequence is also isolated in the first step, if they were present in the mixed sample. Whether MRSA is actually present or not can then be demonstrated in the second step by the detection of S. aureus-specific sequences in the DNA isolated in step a).

Exactly as for the first embodiment described above of the method according to the invention (variant A), it is also possible in the second embodiment (variant B) to use a mixture of genome probes for the isolation of the DNA in step a).

The MRSA nucleotide sequence which is used in step a) for the isolation by means of the genome probe (for example, the mecA gene) can be located in different regions of the SCCmec cassette. In addition, it cannot be ruled out that DNA fragments of differing lengths are isolated owing to the DNA fragmentation which can be expected during DNA isolation. Therefore, there is for example the risk that, although a mecA-containing MRSA DNA fragment is isolated in step a), the S. aureus detection in step b) nevertheless has a negative result because the S. aureus-specific sequence to be detected in step b) is not present in the isolated MRSA fragment owing to, for example, said fragmentation. In order to lower this risk, as per one embodiment of variant B, the DNA isolated in step a) is tested for multiple different specific sequences of S. aureus. Preferably, these different S. aureus-specific sequences are spaced apart, for example on different genome segments. By detecting multiple different S. aureus-specific sequences, the risk of false-negative results is further reduced.

According to one embodiment of variant B of the method according to the invention, the DNA isolated in step a) is tested for S. aureus-specific sequences by means of PCR. Details concerning appropriate PCR detection have already been explained in conjunction with the method according to the invention as per variant A and can be applied analogously to the embodiment as per variant B. Reference is made to the above explanations.

Details concerning possible designs of the genome probes, supports, surface materials, binding of the genome probes to the support, methods for isolating DNA, and further embodiments of the method according to the invention have already been described in conjunction with the method according to the invention as per variant A and can be applied analogously to the embodiment as per variant B. Reference is made to the above explanations.

In a preferred embodiment of the method according to the invention, additionally a control DNA is detected which is specific for a human sequence, in order to ensure that the sample is indeed a human sample.

The sample can be any type of biological sample, more particularly body fluids. In a preferred embodiment, the biological sample is a human sample, and in a particularly preferred embodiment, the sample is a nasal swab from a human patient. As explained, the sample can also be DNA which has already been purified and was recovered from an appropriate biological sample.

The present invention also provides novel primer and probe sequences which enable detection of MRSA. These sequences are as follows:

SauChr
(SEQ ID NO: 1)
TCAATTAACACAACCCGCATCATTTG
saur-sd
(SEQ ID NO: 2)
Fam-CGCATAATCTTAAATGCTCTATACACTTG-BHQ1
type5aw-rev
(SEQ ID NO: 3)
CACTAGTGTAATTATCGAATGATTTATAACTAC
pvl_for
(SEQ ID NO: 4)
TTACACAGTTAAATATGAAGTGAACTGG
pvl_rev
(SEQ ID NO: 5)
CTGCATCAACTGTATTGGATAGC
pvl_sd
(SEQ ID NO: 6)
Hex-AAACTCATGAAATTAAAGTGAAAGGACATAATTGA-BHQ 1

These primers can, inter alia, be used advantageously in the above-described real-time PCR methods.

There are particularly aggressive MRSA strains which, inter alia, are characterized in that they contain Panton-Valentine leukocidin (PVL, or PVL toxin). To detect such MRSA strains, preference is given to using the sequences having SEQ ID NOs:4, 5 and 6.

In a further embodiment, the present invention therefore provides a method for detecting MRSA strains, wherein:
1. chromosomal DNA of S. aureus is isolated from the sample;
2. a nucleotide sequence which is specific for MRSA is detected in the isolated DNA, and
3. the presence of PVL is detected.

In a preferred embodiment, the nucleotide sequence is a resistance gene, and in a particularly preferred embodiment, the resistance gene is mecA.

A further embodiment of the present invention provides a method, wherein:
1. DNA is isolated from a sample, wherein the isolation makes use of a genome probe which is specific for an MRSA nucleotide sequence, preferably an MRSA resistance gene;
2. the DNA isolated in step 1 is tested for specific sequences of S. aureus, and
3. the presence of PVL is detected.

In a preferred embodiment, the resistance gene is mecA.

These methods for detecting MRSA have the advantage that they additionally detect the presence of the PVL toxin, which causes a particularly severe course of disease.

PVL is preferably detected by means of the sequences of SEQ ID NOs: 4, 5 and 6 in the context of real-time PCR.

A further method according to the invention is defined by the use of multi-locus PCR. In this method, multiple loci are detected by means of PCR, which together enable detection of MRSA. Suitable loci are preferably mecA and an S. aureus locus. The PCRs can be carried out as separate PCRs or as multiplex PCR.

In addition, the present invention provides a kit for detecting MRSA in a sample, comprising at least the following components:

Variant A

a) at least one genome probe which is suitable for isolating DNA of S. aureus, and

b) means for detecting a nucleotide sequence which is specific for MRSA; or

Variant B

a) at least one genome probe which is suitable for isolating, from a sample, DNA having an MRSA nucleotide sequence, preferably an MRSA resistance gene, and

b) means for detecting S. aureus-specific DNA sequences.

This kit is suitable especially for carrying out the above-described methods according to the invention. The genome probe present in the kit according to the invention enables the isolation of the target DNA. In addition, the kit comprises means for detecting an MRSA-specific nucleotide sequence (variant A) or means for detecting an S. aureus-specific nucleotide sequence. Suitable means for detecting specific sequences are well known to a person skilled in the art and are not only labeled probes but also, for example, oligonucleotides or oligonucleotide mimetics which enable appropriate detection by means of PCR.

The genome probe as per variant A present in the kit according to the invention enables the isolation of the S. aureus target DNA, and this is advantageous especially for mixed samples. In addition, the kit comprises means for detecting a nucleotide sequence which is specific for MRSA. Details concerning the interaction of the respective elements have already been described above in conjunction with the corresponding method according to the invention (variant A); reference is made to the corresponding embodiments.

According to one embodiment, the kit as per variant A has a genome probe which is complementary to a segment of the chromosomal S. aureus DNA, the segment being specific for S. aureus. This enables the specific isolation of S. aureus DNA from a sample which contains not only S. aureus DNA but also other, non-target DNA.

According to one embodiment, the kit as per variant A has a genome probe which is complementary to a segment of the chromosomal S. aureus DNA which is in the vicinity of the SCCmec insertion site. Preferably, the kit has a genome probe which is complementary to a region within 20 kb, preferably 10 kb, particularly preferably 5 kb, of the SCCmec insertion site. According to a preferred embodiment, the kit comprises a genome probe which has the sequence having SEQ ID NO 7. The kit can further comprise a genome probe which binds within 1 kb, preferably 500 bp, particularly preferably 250 bp, of the region of S. aureus to which the sequence having SEQ ID NO 7 is complementary. The advantages of a corresponding embodiment have already been explained above in conjunction with the method according to the invention as per variant A.

Preferably, the kit as per variant A comprises means for PCR detection of the nucleotide sequence which is specific for MRSA, preferably mecA. As agents for PCR detection of mecA, the kit can comprise at least one primer which has a sequence selected from the sequences having SEQ ID NO 9 to 11. As shown in the example, these enable PCR detection of mecA. The primers as per SEQ ID NO 9 and 10 enable regular PCR detection; the probe as per SEQ ID NO 11 enables, in combination with the other two primers, detection by means of real-time PCR.

The genome probe present in the kit according to the invention as per variant B enables the isolation of the target DNA which has an MRSA-specific nucleotide sequence, preferably a resistance gene such as mecA, and this is advantageous especially for mixed samples. In addition, the kit comprises means for detecting S. aureus-specific DNA sequences. Details concerning the interaction of the respective elements have already been described above in conjunction with the method (variant B); reference is made to the corresponding statements.

According to one embodiment, the kit as per variant B comprises agents for detecting multiple different specific sequences of S. aureus. Preferably, these different S. aureus-specific sequences are spaced apart, for example on different genome segments. The detection of multiple different S. aureus-specific sequences further reduces the risk of false-negative results, as explained above in conjunction with the method according to the invention as per variant B. Reference is made to the above disclosure.

Preferably, the kit as per variant B comprises means for PCR detection of the S. aureus-specific sequences.

According to one embodiment, the kit comprises a genome probe which is ≧20 nucleotides and preferably ≦100 nucleotides in length.

The kit can further comprise multiple different genome probes. Advantages have been explained in conjunction with the methods according to the invention; reference is made to the above statements.

According to a preferred embodiment, the kit comprises a support for binding the genome probe. In addition, the genome probe can be present bound to the support. Details and advantages have been explained in conjunction with the methods according to the invention; reference is made to the above statements.

Preferably, the kit comprises oligonucleotides or oligonucleotide mimetics for detecting the specific sequences. Details and advantages concerning this have been explained in conjunction with the methods according to the invention; reference is made to the above statements.

### EXAMPLE

The method according to the invention as per variant A is illustrated with the aid of the following exemplary embodiment. This describes only one possible embodiment of the invention and is therefore non-limiting. Analogous methods can be used to carry out the method as per variant B.

I. Material

The following materials were used:

1. Dynabeads kilobaseBINDER Kit (Invitrogen, cat. no. 601.01).

2. A genome probe for isolating chromosomal DNA of S. aureus having the following sequence:

sa_fish:
(SEQ. ID NO 8)
Biotin-tatcctatcctatcctgATGAAAGCTTTATTACTTAAAACA
AGTGTATGGCTCGTTTTGCTTTTTAGTGTAATGGGATTATGGCAAG

The region in lower case at the beginning of the probe is artificial and serves only as a spacer to the biotin and, thus later, to the support to which the genome probe is bound. The S. aureus-specific sequence of the genome probe was designed based on the following database entry:
LOCUS CP000255 2319 by DNA linear BCT 12-MAR-2009
DEFINITION staphylococcus aureus subsp. aureus USA300_FPR3757, complete genome.
ACCESSION CP000255 REGION: 31026 . . . 33344

3. A Dynal magnet

4. A vortexer

5. Pipets

6. Roller mixer

7. NaOH, 0.125 M (fresh!)-“melting solution”

8. 20% acetic acid

II. Purification of the S. aureus-specific DNA

The experiment was carried out based on the following protocol:
The Dynabeads are resuspended by shaking or vortexing the vial in order to obtain a homogeneous suspension.
2. 5 μl (50 μg) of the resuspended beads are transferred to a 1.5 ml microcentrifuge tube. The tubes are placed over the magnet for 2 minutes (or until all beads have migrated to the side).
3. The supernatant is carefully removed by pipetting while the tube remains on the magnet. Contact between the bead pellets and the pipet tip should be avoided.
4. Remove the tube from the magnet. 20 μl of Binding Solution (component of the Dynabeads kilobaseBINDER Kit) are added at the inner side of the tube, where the beads have gathered, and the beads are gently resuspended using the pipet. The solution may be viscous.
5. The tubes are again placed on the magnet and the supernatant (Binding Solution) is removed as in step 3 above.
6. The beads are resuspended in 20 μl of Binding Solution.
7. 350 pmol (70 pmol per 1 μg of beads) of the genome probe (see above) are added to 10⁵ genome copies of S. aureus. For this purpose, use was made of 20 μl of an appropriate sample containing S. aureus DNA, dilution 1:100. In this step, the genome probe attaches to the DNA. For the purposes of this experiment, use was made of purified S. aureus DNA in order to show that the genome probes can bind and isolate S. aureus DNA.
8. Incubation of the tube on a roller mixer for 1 hour at room temperature (about 15-25° C.), so that the beads remain in suspension.
9. 20 μl of the bead suspension are added to the sample.
10. Incubation of the tube on a roller mixer for 1 hour (20 min) at room temperature (15-25° C.) in order to keep the beads in suspension. In this step, the beads bind to the biotin of the genome probe. As a result, the genome probe (with the bound S. aureus-specific DNA) attaches to the beads.
11. The tubes are placed on the magnet and the supernatant is removed as described in step 3.
12. The Dynabeads/DNA complex is washed twice in 40 μl of the Washing Solution (component of the Dynabeads kilobaseBINDER Kit) and once in distilled H₂O or Tris-HCl, pH 8.0.
13. Completely remove water/buffer.
14. Preparation of the neutralization solution by mixing 500 μl of PB and 3.8 μl of 20% acetic acid.
15. Add 50 μl of the “melting solution” (0.125 M NaOH) to the beads.
16. Vortex.
17. Remove supernatant by means of a Magnetic Particle Concentrator (MPC).
18. The supernatant is added to a tube containing neutralization solution.
19. Steps 15 to 18 are repeated.
20. The melted sample is transferred to a spin column, centrifuged, and the flow-through is discarded. 750 μl of PE is added and spun down again. The flow-through is discarded, the tube is rotated 180° and centrifuged again. The DNA is eluted using 15 μl of TE buffer.

III. Detection of the mecA Resistance Gene

The following real-time PCR was carried out in order to detect the mecA resistance gene in the sample and thus MRSA in the S. aureus DNA isolated earlier.

The PCR was prepared as follows:

| | | | Final | | Amount/ | |
|---|---|---|---|---|---|---|
| Component | Concentration | | concentration | | reaction | |
| Real-time | 5X | | 1X | | 5 | μl |
| PCR buffer | | | | | | |
| dNTPs | 10 | mM | 0.14 | mM | 0.35 | μl |
| BSA | 20 | mg/ml | 0.2 | mg/ml | 0.25 | μl |
| HotStarTaq | 5 | U/μl | 0.192 | U/μl | 0.96 | μl |
| MgCl₂ | 200 | mM | 5 | mM | 0.625 | μl |
| mec_for (21) | 100 | μM | 0.5 | μM | 0.125 | μl |
| mec_rev (22) | 100 | μM | 0.5 | μM | 0.125 | μl |
| mec_sd2 | 100 | μM | 0.05 | μM | 0.0125 | μl |
| (54) | | | | | | |
| H₂O | | | | | 12.5525 | μl |
| Template | | | | | 5.00 | μl |
| | | | | | 25 | μl |

Cycler

program

| | | | |
|---|---|---|---|
| 95° C. | 15 minutes | Green | mec- |
| | | | primer |
| 95° C. | 20 seconds | Orange | PVL |
| 53° C. | 20 seconds | Crimson | Nuc |
| 72° C. | 30 seconds | | |
| 45 cycles | | | |

For the detection of the mecA resistance gene by means of real-time PCR, the following primers/samples were used:

mec_for
(SEQ ID NO 9)
ATTACCGTTCTCATATAGCTCATCATAC
mec_rev
(SEQ ID NO 10)
ATAAAGATAATCCAAACATGATGATGGC
mec_sd2
(SEQ ID NO 11)
FAM-CCATTCCTTTATCTTGTACATCTTTAACATT-BHQ1

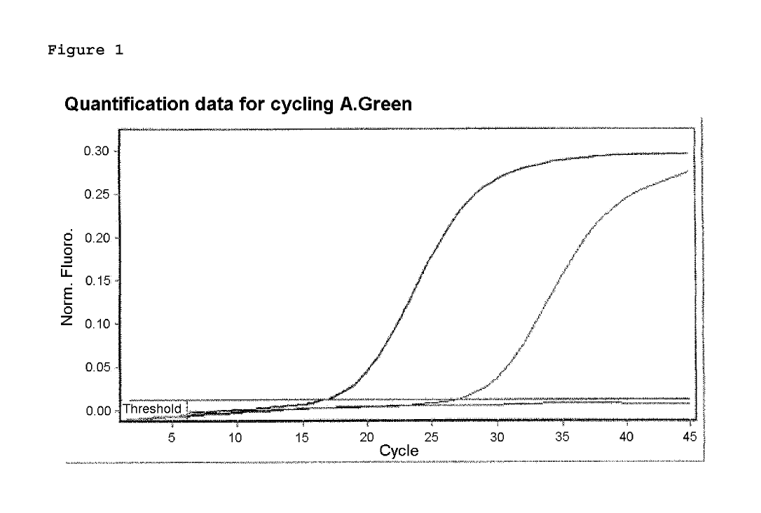

The results of the PCR are shown in FIG. 1. Displayed on the left is the curve in which the original sample, i.e., the S. aureus DNA purified by means of a conventional method, was used as template (there was accordingly no specific isolation by means of the genome probe). Displayed on the right is the curve in which the template used was the S. aureus DNA purified according to the above-described method using the genome probe. The results show that the genome probes make it possible to isolate S. aureus and also made it possible to detect mecA. Accordingly, the method would also be suitable for isolating S. aureus from a mixed sample in which not only S. aureus DNA but also other, non-target DNAs are present.

1. A method for detecting MRSA in a sample comprising:
per variant A,
a) isolating chromosomal DNA of S. aureus from the sample by means using a genome probe, and
b) determining the presence or absence of a nucleotide sequence specific for MRSA in the chromosomal DNA isolated in step a). wherein the presence of the nucleotide sequence indicates the presence of MRSA in the sample;
or
per variant B,
a) isolating DNA from the sample using a genome probe specific for an MRSA nucleotide sequence, and
b) determining the presence or absence of a specific sequence of S. aureus in the DNA isolated in step a), wherein the presence of the specific sequence of S. aureus indicates the presence of MRSA in the sample.

2. The method according to claim 1, wherein when per variant A, the genome probe is complementary to a segment of the chromosomal DNA, which segment is specific for S. aureus.

3. The method according to claim 1, wherein when per variant A,
a) the genome probe is complementary to a segment of the chromosomal S. aureus DNA in the vicinity of the SCCmec insertion site;
b) the genome probe is complementary to a segment of the chromosomal S. aureus DNA within a region of 25 kb to the SCCmec insertion site;
c) the genome probe is complementary to a segment of the chromosomal S. aureus DNA that is in the vicinity of the mecA gene and is S. aureus-specific;
d) the genome probe is complementary to a segment of the chromosomal S. aureus DNA that is within a region of 25 kb of the mecA gene and is S. aureus-specific; and/or
e) the genome probe comprises a sequence as set forth in SEQ ID NO:7 or is a genome probe complementary to a segment of the chromosomal S. aureus DNA within 1 kb of the region of the S. aureus chromosome to which the sequence having SEQ ID NO:7 is complementary.

4. The method according to claim 1, wherein
a) the genome probe is ≧20 nucleotides in length;
b) the genome probe is bound to a support;
c) the genome probe is bound to the support via a spacer;
d) the genome probe is present covalently bonded to a support;
e) the genome probe is contacted with the DNA in a first step and the support is added in a second step;
f) the genome probe is present bound to the support before it is contacted with the DNA; and/or
g) when per variant A, multiple different genome probes are used to isolate the chromosomal DNA of S. aureus , or when per variant B, a mixture of genome probes are used to isolate the DNA.

5. The method according to claim 1, wherein when per variant A, the nucleotide sequence is a resistance gene.

6. The method according to claim 1, wherein when per variant A, the presence or absence of the nucleotide sequence specific for MRSA is determined by means of PCR; or wherein when per variant B, the presence or absence of the specific sequence of S. aureus in the DNA isolated in step a) is determined by means of PCR.

7. The method according to claim 1, wherein when per variant B, the presence or absence of multiple different specific sequences of S. aureus in the DNA isolated in step a) is determined.

8. The method according to claim 1, wherein DNA is isolated from the sample in a preceding step
(i) before the chromosomal S. aureus DNA is isolated using the genome probe in step a) when per variant A, or
(ii) before the DNA is isolated by means of the genome probe specific for an MRSA nucleotide sequence in step a) when per variant B.

9. The method according to claim 1, wherein the sample is a mixed sample.

10. The method according to claim 1, further comprising determining the presence or absence of the Panton-Valentine leukocidin (PVL) gene.

11. The method according to claim 1, further comprising detecting the presence or absence of a control DNA specific for a human sequence.

12. A kit for detecting MRSA in a sample, comprising:
per variant A,
a) at least one genome probe suitable for isolating DNA of S. aureus, and
b) means for detecting a nucleotide sequence is specific for MRSA;
or
per variant B,
a) at least one genome probe suitable for isolating from a sample DNA comprising an MRSA resistance gene, and
b) means for detecting an S. aureus-specific DNA sequence.

13. The kit according to claim 12, wherein per variant A,
a) the kit comprises a genome probe complementary to a segment of the chromosomal S. aureus DNA, which segment is specific for S. aureus;
b) the kit comprises a genome probe complementary to a segment of the chromosomal S. aureus DNA in the vicinity of the SCCmec insertion site;
c) the kit comprises a genome probe complementary to a region within 20 kb of the SCCmec insertion site;
d) the kit comprises a genome probe containing the sequence having SEQ ID NO:7 or has a genome probe that binds within 1 kb of the region of S. aureus to which the sequence having SEQ ID NO:7 is complementary;
e) the kit comprises means for the PCR detection of the nucleotide sequence specific for MRSA; and/or
(f) the kit comprises means for PCR detection of mecA, at least one primer comprising a sequence selected from the sequences having SEQ ID NOS:9 to 11.

14. The kit according to claim 12, wherein when per variant B, the kit comprises
a) means for detecting multiple different specific sequences of S. aureus; and/or
b) means for PCR detection of the S. aureus-specific sequences.

15. The kit according to claim 12, wherein
a) the kit comprises a genome probe that is ≧20 nucleotides;
b) the kit comprises multiple different genome probes;
c) the kit comprises a support for binding the genome probe or the genome probe is present bound to the support; and/or
d) the kit comprises oligonucleotides or oligonucleotide mimetics for detecting the specific sequences.


SEQUENCE LISTING (11 sequences)

SEQ ID NO: 1 - DNA, 26 bp, Artificial Sequence
  Primer SauChr
tcaattaacacaacccgcatcatttg

SEQ ID NO: 2 - DNA, 29 bp, Artificial Sequence
  Primer probe saur-sd
cgcataatcttaaatgctctatacacttg

SEQ ID NO: 3 - DNA, 33 bp, Artificial Sequence
  Primer type5aw-rev
cactagtgtaattatcgaatgatttataactac

SEQ ID NO: 4 - DNA, 28 bp, Artificial Sequence
  Primer pvl_for
ttacacagttaaatatgaagtgaactgg

SEQ ID NO: 5 - DNA, 23 bp, Artificial Sequence
  Primer pvl_rev
ctgcatcaactgtattggatagc

SEQ ID NO: 6 - DNA, 35 bp, Artificial Sequence
  Primer probe pvl_sd
aaactcatgaaattaaagtgaaaggacataattga

SEQ ID NO: 7 - DNA, 70 bp, Artificial Sequence
  Genome probe for S. aureus
atgaaagctttattacttaaaacaagtgtatggctcgttttgctttttagtgtaatgggattatggcaag

SEQ ID NO: 8 - DNA, 87 bp, Artificial Sequence
  Genome probe for S. aureus
tatcctatcctatcctgatgaaagctttattacttaaaacaagtgtatggctcgttttgctttttagtgtaatgggattatggcaag

SEQ ID NO: 9 - DNA, 28 bp, Artificial Sequence
  Primer mec_for, for  detection of the mecA gene detection
attaccgttctcatatagctcatcatac

SEQ ID NO: 10 - DNA, 28 bp, Artificial Sequence
  Primer mec_rev, for detection of the mecA gene detection
ataaagataatccaaacatgatgatggc

SEQ ID NO: 11 - DNA, 31 bp, Artificial Sequence
  Primer probe mec_sd2 for real-time PCR for the detection of the mecA gene
ccattcctttatcttgtacatctttaacatt